(19) United States
(12) Patent Application Publication
(10) Pub. No.: US 2003/0008839 A1
(43) Pub. Date: Jan. 9, 2003

(54) Adjuvants for nucleic acid vaccines

(75) Inventors: Eugene Marie, A. van Rooij, Lelystad (NL); Lucas Alfonsus, T. Hilgers, Utrecht (NL)

Correspondence Address: BANNER & WITCOFF, LTD., 28 STATE STREET, 28th FLOOR, BOSTON, MA, 02109, US

(73) Assignee: I-D Lelystad, Instituut voor Dierhouderijen, Lelystad (NL)

(21) Appl. No.: 10/128,148

(22) Filed: Apr. 23, 2002

Related U.S. Application Data
(63) Continuation of application No. PCT/NL00/00773, filed on Oct. 26, 2000.

(30) Foreign Application Priority Data
Oct. 26, 1999 (EP) 99203522.0

(51) Int. Cl. A61K 48/00; A61K 31/14

(52) U.S. Cl. 514/44; 514/642; 424/93.2

(57) ABSTRACT

The present invention relates to the field of vaccination. In particular, the invention relates to immunization by administration of a nucleic acid encoding a gene produce (e.g., proteins, glycoproteins, lipoproteins) of an infectious or non-infectious agent. In accordance with the invention, it has been found that dimethyldialkylammonium salts enhance the immune response of a nucleic acid vaccine.

The invention relates to the field of vaccination; both for prophylactic and therapeutic use (e.g. in the area of infectious diseases, cancer/tumorology, auto-immunity or endocrinology). In particular, the invention relates to immunization by administration of nucleic acids encoding gene products (e.g. proteins, glycoproteins, lipoproteins) of infectious or non-infectious agents.

In the field of immunization, adjuvants have been used to improve vaccine efficacy from the early 1920s. Classically, adjuvants are selected for the ability to generate a, preferably protective, immune response. Adjuvants are to improve the uptake of antigens by the immune system, and stimulate antigen-presenting cells (APC) to express certain signals, such as the secretion of cytokines. While the number of substances with adjuvant activity and the literature describing their use has expanded enormously, their mode of action has remained largely mysterious and empirical.

In Res. Immunology 143, 494-503, 1992, Hilgers et al. have described the use of dimethyldioctadecylammonium bromide 1(DDA) as an effective adjuvant for both humoral and cell-mediated immune responses. It was proposed to use DDA in experimental vaccines and in commercial vaccines for veterinary purposes, especially if cell-mediated immunity is considered of relevance.

However, in spite of the multitude of available options disclosed in the literature, only aluminum salts have gained acceptance as human vaccine adjuvants and even veterinary vaccines are largely dependent on the use of aluminum salts.

Within the field of immunization and vaccination, nucleic acid immunization holds a special and distinct place. According to this technique, plasmid nucleic acid encoding appropriate genes is directly inoculated into the vaccinee. The nucleic acid is to be taken up by cells, by an as yet ill-explained mechanism, and transported into the cell compartment of interest (e.g. for DNA: transported to the nucleus where transcription into mRNA occurs) with subsequent production of the encoded proteins (e.g. (glyco)proteins). Although expression in specific cell types has been clearly demonstrated, the cell type responsible for inducing the immune response remains unidentified.

In an attempt to optimize the immune responses induced by nucleic acid vaccination, studies have been undertaken to evaluate the various vehicles in which nucleic acid may be delivered, and the routes by which the nucleic acid may be administered. Nucleic acid may be administered naked, e.g. dissolved in a saline solution, complexed with lipids, or dried on the surface of microscopic beads. It may be inoculated by various routes, including intravenous, intraperitoneal, intramuscular, intradermal, intranasal and biolistic. It has even been suggested that it could be feasible to apply it topically, simply by rubbing nucleic acid onto skin.

Recently, it has been recognized that naked nucleic acid encoding for relevant protein antigens of an infectious agent can be used to confer protective immunity by activating humoral and cell-mediated immunity. The efficacy of these nucleic acid vaccines is, however, in general insufficient to establish long-lasting protective immunity. The efficacy of nucleic acid vaccines is often relatively low, therefore they need adjuvants or vehicles that induce or enhance an immune response.

Typically, an adjuvant for use in nucleic acid vaccination should fulfill two functions. On the one hand, it should assist in transporting the nucleic acid into cells and in allowing the nucleic acid to become expressed On the other hand, it should preferably assist in inducing an immune response.

In FEBS Letters 402 (1997), 107-110, and in AIDS Research and Human Retroviruses, vol. 13, no. 16, 1997, 1421-1428, it has been proposed to use cationic liposomes as adjuvant for nucleic acid vaccination. Although it was reported that liposomes are of assistance in the induction of humoral and cell mediated immune responses, their assistance in transporting the nucleic acid into cells and allowing to come to expression leaves room for improvement.

The present invention seeks to provide an improved adjuvant for nucleic acid vaccination. In particular, it is desired that the objective adjuvant assists in processes required for an optimal stimulation of an immune response. It is furthermore desired that an adjuvant is provided which enables an advantageous transport of a nucleic acid into cells, resulting in the production of the desired antigen coded for by the nucleic acid.

It has now surprisingly been found, that certain specific ammonium salts are particularly suitable as adjuvant for nucleic acid vaccination or immunization. More in particular, it has been found that dimethyldialkylammonium salts enhance the transport of nucleic acid administered to a vaccinee into cells and facilitates expression of said nucleic acid to produce desired antigen. Furthermore, it has been found that dimethylalkylammonium salts assist in inducing an immune response in the vaccinee.

The anion in the dimethyldialkylammonium salts that are used as adjuvants in accordance with the invention are preferably halogen ions. Particularly good results have been obtained with iodide, bromide or chloride salts.

In a preferred embodiment, the two alkyl groups of the dimethyldialkylammonium salts are independently chosen from the group of saturated or unsaturated aliphatic alkyl chains having from 12 to 24, more preferably from 14 to 20, carbon atoms. Most preferred is the use of a dimethyl-dioctadecylammonium salt.

The present adjuvants may be formulated in any type of nucleic acid vaccine wherein the nucleic acid is capable of being expressed after vaccination to yield a specific desired antigen. The nucleic acid may be a DNA, cDNA, positive or negative stranded RNA or mRNA molecule or a combination thereof. If the nucleic acid is an RNA molecule, it is preferably non-ribosomal.

The nucleic acid may encode a gene product of an infectious agent, such as viruses, bacteria, mycoplasms, helminths, protozoa, or prions, or a non-infectious agent, such as hormones, enzymes or cytokines. Examples of pathogens include influenzaviruses, HIV, hepatitis viruses, herpesviruses, pestiviruses, flaviviruses, reproductive and respiratory syndrome viruses, mycobacteria, streptococci, Borrelia, mycoplasma pulmonis, malaria-plasmodium and trypanosomiasis. This list of examples of pathogens is not exhaustive; the skilled person will be able to identify many more suitable pathogens, which will all fall within the scope of the invention. The vaccine may be for prophylactic or therapeutic purposes in mammals, poultry, fish, amphibians or reptiles.

It may be employed in a naked form, but also using bacterial plasmid vectors or replication defective viral or bacterial delivery systems. Examples of viral delivery systems are Semliki forest virus and Sindbis virus based expression systems. Examples of bacterial delivery systems are gram negative and/or positive bacteria such as Shigella flexneri, Salmonella typhimurium, and Listeria monocytogenes. This list of examples of delivery systems is not exhaustive; the skilled person will be able to identify many more suitable delivery systems, which will all fall within the scope of the invention.

Often, the nucleic acid will be dissolved in a suitable solvent, such as a buffer, prior to formulation with the dimethyldialkyl ammonium salt. Many suitable buffers in this regard are known to the skilled person, such as tris based buffers or phosphate buffers, such as PBS. It is preferred, especially when the vaccine is to comprise relatively small amounts of the dimethyldialkyl ammonium salt, that the buffer used does not contain multivalent anions. In case larger amounts of the dimethyldialkyl ammonium salt are used, the effect of the presence of multivalent anions will be less noticeable.

It is to be noted that in U.S. Pat. No. 5,951,988, the possibility of using a quaternary ammonium salt as an adjuvant for a DNA containing vaccine has been suggested. For practical purposes, however, this document only discloses the use of quaternary ammonium salts as adjuvant for ordinary, antigen containing vaccines. This is partly apparent from the fact that the document teaches that it is essential that the quaternary ammonium salt is used in the form of an oil containing emulsion, since mineral oils are commonly used in ordinary, antigen containing vaccines.

In accordance with the present invention, it has surprisingly been found that the presence of an oil inhibits the action of dimethylalkylammonium salts as an adjuvant for nucleic acid vaccines. As has been disclosed in the aforementioned U.S. Pat. No. 5,951,988, it is difficult to formulate stable emulsions of quaternary ammonium salts in this context. It is often necessary to include a detergent in order to increase the stability of the emulsion. These detergents may cause or ameliorate local and systemic side-effects of a vaccine. Even more importantly, the oil (droplets), the detergent, or both have been found to negatively interfere with the adjuvant activity of the dimethylalkylammonium salt, in that they may bind physically to the dimethylalkylammonium salt or to a complex of the dimethylalkylammonium salt and a nucleic acid. As a result of this, the adjuvant activity of the dimethylalkylammonium salt and the immune response of the vaccine will be negatively affected. The binding of a nucleic acid to a dimethylalkylammonium salt located on the surface of oil droplets further causes large, nucleic acid coated particles, which are difficult to process.

For these and other reasons, it is preferred that a dimethylalkylammonium salt is used in the absence of an oil and not in the form of an emulsion. Even more preferred, is the use of a dimethylalkylammonium salt in a pharmaceutically acceptable aqueous solvent or buffer, which preferably substantially does not contain multivalent anions, such as phosphate ions. The aqueous solvent can be an ionic isotonic solvent such as a solution of sucrose in water-for-injection. The pH of the solvent is preferably in the range applicable to pharmaceutically acceptable products, e.g. between 6.8 and 7.3. Of course, the same considerations apply to a vaccine containing a dimethylalkylammonium salt according to the invention.

In a preferred embodiment, the nucleic acid vaccine is a solution of a salt, e.g. saline, and a buffer, comprising the adjuvant and the nucleic acid. Of course it is also possible to use a vaccine wherein the nucleic acid is immobilized on a carrier, such as an inert particle or a liposome. However, care should be taken that a carrier is used that is not too hydrophobic and has a suitable size and surface, in order to avoid the problems associated with the use of an oil emulsion as disclosed in the above mentioned U.S. Pat. No. 5,95,988. Suitable immobilization techniques are known per se. Preferably, the solution comprises between 0.5 and 32 mg, more preferably between 1 and 16 mg, particularly between 6 and 12 mg, adjuvant per ml solution. Further, it is preferred that the solution comprises between 0.05 and 2 mg nucleic acid per ml. The nucleic acid dose per vaccination should preferably be between 0.001 and 2000 μg. The salt and buffer concentrations of the solution depend on the envisaged application of the vaccine. Choosing suitable concentrations for the salt and the buffer is well within the skills of the skilled person.

In accordance with the invention, the vaccine may be administered in any known manner. Suitable examples of administration methods include intravenous, intraperitoneal, intramuscular, intradermal, intranasal and biolistic administration. Preferred manners of administration are by syringe injection, using air pressure devices, e.g. based on air or helium, or topical administration, e.g. with or without the use of dimethylsulfoxide (DMSO).

The invention will now be elucidated by the following, non-restrictive examples.

### EXAMPLES

Nucleic Acid Preparation

The full-length gD gene and gB gene were cloned into vector VR1012 according to the following procedure.

For gD, a Hind III/Eco RI fragment from plasmid pMZ33, containing the full length gD, was cloned into vector VR1012 (Vical, San Diego, USA). For gB, a Hind III/Bam HI fragment from plasmid pUC19-gB, which contains the full length gB gene was cloned into VR1012. Plasmid VR1012 contains the human cytomegalovirus immediate early promoter, intron A, the processing signal for bovine growth hormone polyadenylation and the gene encoding kanamycin resistance. and characterized by restriction mapping. As negative control served the plasmid which contained no insert. Plasmids were grown in the HB101 strain of Escherichia coli and purified on Qiagen columns (Qiagen GmbH, Germany) and stored at a concentration of 1 mg plasmid DNA/ml PBS at −20° C. prior to use. Expression of recombinant proteins was checked as described previously (Van Rooij et al., 1998).

Adjuvant Formulation

Dimethyldioctadecylammonium bromide (Aldrich) was dissolved in saline by heating the suspension at 60° C. for 10 min while stirring the suspension.

DNA+DDA Formulation

The DNA preparation and the DDA formulation were mixed at appropriate volume ratio.

Animal Experiments

Ten- to twelve-week old Dutch landrace pigs and Minnesota miniature pigs from the specified-pathogen-free herd of the Institute for Animal Science and Health (ID-DLO) were used. Inbred Minnesota miniature pigs, congeneic for the swine-leucocyte-antigen complex (SLA) haplotype d/d (Sachs et al., 1976), were used to measure MHC restricted CTL responses. All pigs were born from unvaccinated sows, were free from antibodies against PRV before the start of the experiment, and were randomly allocated to the experimental groups. Experimental procedures and animal management procedures were undertaken in accordance with the requirements of the animal care and ethics committees of the institute according to the Dutch legislation on animal experiments.

Three groups of 5 pigs (4 outbred landrace pigs and 1 inbred Minnesota miniature pig per pig) were injected with the DNA-DDA formulation three times at intervals of 4 weeks. Pigs were vaccinated intradermally in the lateral side of the neck behind the ear by way of a 22-gauge needle.

Group I received 400 μg gB+400 μg gD per dose of 2 ml.

Group II received 400 μg gB+400 μg gD+16 mg DDA per dose of 2 ml.

Group III received 800 μq of empty control plasmid per dose of 2 ml.

At weekly intervals, starting one week before first immunization, blood samples for serum and peripheral blood mononuclear cells (PBMC) were collected to assess the presence of virus neutralizing (VN) antibodies and cell-mediated immune responses.

Six weeks after the third immunization, all pigs were challenged intranasally with 10⁵ plaque-forming units (pfu) of virulent, wildtype pseudorabies virus strain NIA-3 (MacFerran & Dow, 1975) per animal prepared on secondary porcine kidney cells as described by Kimman et al. (1992). Antibody and cell-mediated immune responses were measured until 21 days after challenge. Clinical signs (e.g. listlessness, loss of appetite, nasal discharge, coughing, ataxia, convulsions, paralysis, death), rectal temperatures and body weights were recorded for 14 days after challenge. Pigs were deemed to have fever when body temperatures were above or equal to 40° C. Growth performance was assessed by calculating the mean relative daily gain (MRDG) in body weight according to Stellman et al. (1989). Virus excretion was monitored by collecting swab specimens of oropharyngeal fluid (OFF) from the day before challenge until day 10 after challenge. Swab specimens were extracted with 4 ml of Dulbecco's minimal essential medium (DMEM) supplemented with 2% foetal bovine serum and antibiotics. To determine the virus content per gram OPF, we measured the weight of the collected fluid after centrifuging the swabs.

All pigs injected with gB+gD with or without DDA survived the challenge infection whereas one of the five sham-vaccinated pigs died from PRV infection (see Table 1). Pigs vaccinated with gB+gD with or without DDA showed less severe (some listlessness and loss of appetite) and for a significantly shorter (P<0.05) period of time clinical signs than pigs the sham-vaccinated pigs (Table 1). Pigs vaccinated with gB+gD+DDA were best protected as they showed for a significantly shorter (P<0.05) period of time clinical signs and fever than pigs vaccinated with gB+gD only or the sham-vaccinated pigs. In addition, pigs vaccinated with gB+gD+DDA were better protected as they had a better growth performance resulting in a significantly higher (P<0.05) MRDG7 of 0.9% as compared to pigs vaccinated with the cocktail vaccine only (MRDG7=0.1%) or the sham-vaccinated pigs ((MRDG7 =−1.1%). As a result, pigs vaccinated with gB+gD+DDA had a ΔG of 2 meeting the requirements of the European Pharmacopea.

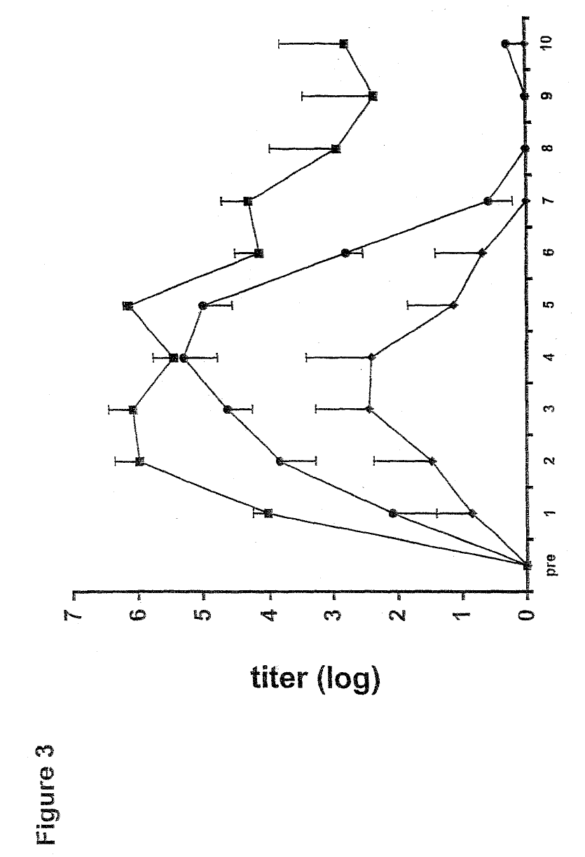

After challenge infection, pigs immunized with the gB+gD+DDA excreted virus for a significantly shorter (P<0.05) period of time than the pigs immunized with gB+gD or the sham-treated pigs (Table 1). In addition, pigs immunized with gB+gD+DDA showed significantly (P<0.05) lower peak levels of virus excretion than pigs immunized with gB+gD only or the sham-treated pigs (FIG. 3).

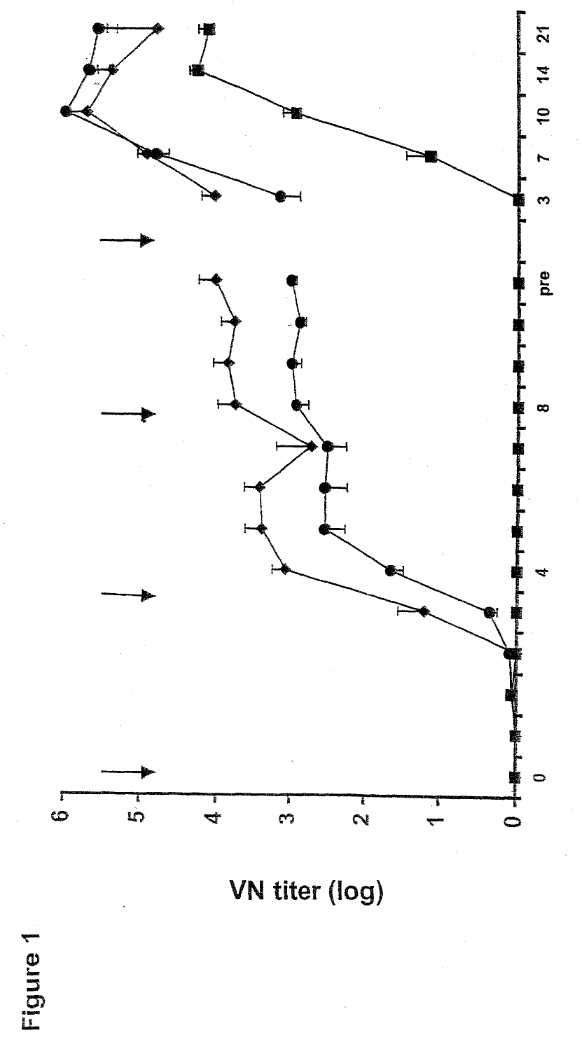

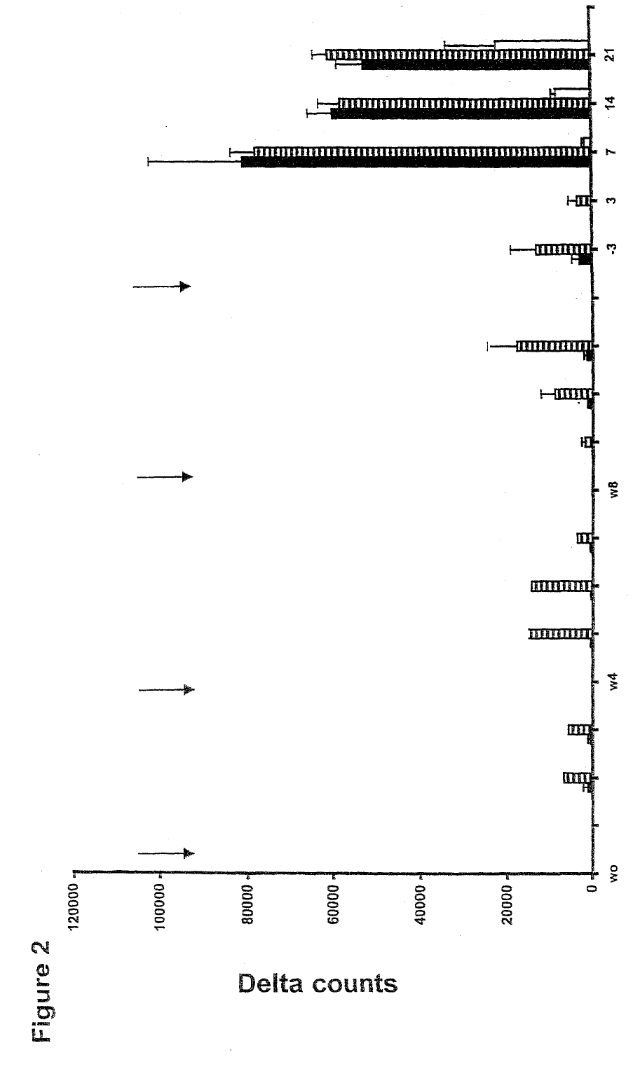

After challenge infection, all groups of pigs seroconverted between days 3 and 7 and pigs vaccinated with the cocktail with or without DDA reached comparable titers of VN antibody titers (FIG. 1). At day 3 after challenge infection, both pigs vaccinated with the cocktail with or without DDA showed a decrease in LPT responses although pigs vaccinated with the cocktail plus DDA still showed an significant higher (P<0.05) LPT response than pigs vaccinated with the cocktail only or the sham-vaccinated pigs. From day 7 onwards, LPT responses increased in all groups of pigs whereby the magnitude of the LPT responses was similar for pigs vaccinated with the cocktail with or without adjuvant and significantly higher (P<0.05) than the LPT responses of the sham-vaccinated pigs. (FIG. 2).

Lymphocyte Proliferation Assay (LPT)

PBMC from pigs were analyzed for PRV specific LPT responses as described by Kimman et al. (1995). Briefly, PBMC were isolated from heparinized blood samples by density gradient centrifugation. The isolated PBMC were seeded in 96-well flat-bottom plates (M29, Greiner, The Netherlands) at a density of 5×10⁶ cells/ml in RPMI 1640 medium (RPMI 1640 containing 10% porcine serum, 2 mM L-glutamine, 50 mM betamercapto-ethanol, 200 U/ml penicillin, 200 mg/ml streptomycin, and 100 U/ml mycostatin). To 100 ml of leukocyte suspension/well, 100 ml of live virus preparation containing 5×10⁶ pfu/ml of the PRV strain NIA-3 was added (four wells per variable). Control wells were incubated with a control sample prepared from non-infected cells (mock control). In each test, four wells were incubated with 5 mg/ml of Con A as vitality control. After 4 days incubation in a humidified incubator at 37° C. in a 5% CO₂ atmosphere, the cultures were pulsed with 0.4 mCi [³H]-thymidine (Amersham, The Netherlands). After 4 h of incubation, cells were harvested, and the incorpo-rated radioactivity was measured in a Betaplate scintillation counter (Wallac, EG&G Instruments, The Netherlands). Proliferation was expressed as the number of counts (mean of quadruplicate wells) of PRV-stimulated PBMC minus the number of counts of the mock control-stimulated PBMC (delta counts). Assays were accepted provided that standard errors of counts of the quadruplicates were less than 20%.

Pigs vaccinated with the cocktail plus DDA developed significantly stronger LPT responses (P<0.05) after second and third vaccination than pigs with the cocktail alone (FIG. 2). Both groups of pigs vaccinated with or without DDA developed significantly stronger LPT responses (P<0.05) than sham-vaccinated pigs throughout the vaccination period.

PRV Neutralising Antibodies

Virus neutralizing (VN) antibodies were detected by incubating sera (in duplicate) with 100 (range: 30-300) tissue infective doses (TCID₅₀) of PRV strain NIA-3 for 24 h at 37° C. (Bitsch & Eskildsen, 1975). Titers are expressed as ¹⁰clog of the reciprocal of the highest serum dilution inhibiting cytopathogenic effect in 50% of the cultures. Before the sera were tested, they were heat-treated for 30 mm at 56° C. to inactivate complement.

VN antibodies were detected from week 3 after the first vaccination (FIG. 1) in pigs vaccinated with the DNA vaccine cocktail with or without DDA. Pigs vaccinated with the cocktail plus DDA developed significantly higher titers (P<0.05) after second and third vaccination than pigs vaccinated with the cocktail alone. Both groups of pigs vaccinated with or without DDA developed significantly higher titers (P<0.05) than sham-vaccinated pigs throughout the vaccination period.

Virus Titration.

The amount of virus excretion was quantitated by titrating the virus on SK-6 monolayers in DMEM supplemented with 5% foetal bovine serum, L-glutamine (0.3 mg/ml), penicillin (90 U/ml), streptomycin (100 U/ml), and nystatin (45 U/ml) in a humidified incubator at 37° C. with 5% CO₂, as described by Kimman et al. (1992).

Statistical Analysis

Differences in LPT responses, VN antibody titers and MRDG were tested for statistical significance by analysis of variance (ANOVA). Differences in levels of virus excretion were tested for statistical significance by the Two-Sample T test. Differences in duration of virus shedding, fever and clinical signs were tested for statistical significance by the non-parametric Kruskal-Wallis test Wardlaw, 1985). The significance level was set at 95.

Legends of Figures

FIG. 1. Virus neutralising (VN) antibody titer in serum of pigs vaccinated with the DNA cocktail (gB+gD) alone (●), the DNA cocktail with DDA (♦), or control plasmid (▪). Pigs (n=5) were vaccinated at weeks 0 (w0), 4 (w4) and (w8) and challenged 6 weeks after third vaccination (arrows). Pigs were sampled weekly after vaccination and at day 3 before (D−3) and at days 3, 7, 10 and 14 after challenge infection. Data are expressed as geometric mean ¹⁰log VN titer (g standard error of mean) of the different groups.

FIG. 2. Cell-mediated, lymphocyte proliferation responses from PBMC of pigs vaccinated with plasmid coding for DNA cocktail (gB+gD) alone (black bar), the DNA cocktail plus DDA (hatched bar) or control plasmid (white bar). Pigs (n=5) were vaccinated at weeks 0 (w0), 4 (w4) and (w8) and challenged 6 weeks after third vaccination (arrows). Pigs were sampled weekly after vaccination and at day 3 before (D−3) and at days 3, 7, 10 and 14 after challenge infection. Data are expressed as arithmetic mean delta counts (+/−standard error of the mean) of the different groups. Delta counts represent the number of counts (mean of quadruplicate wells) of PRV-stimulated PBMC minus the number of counts of the mock control-stimulated PBMC.

FIG. 3. Virus excretion after challenge infection with PRV strain NIA-3 in pigs vaccinated with pigs vaccinated with the DNA cocktail alone (●), DNA cocktail with DDA (♦), or control plasmid (▪). Data are expressed as arithmetic mean ¹⁰log virus titer per gram oropharyngeal fluid (OPF) of the different groups.

### REFERENCES

Bitsch, V. & Eskildsen, M. (1976). A comparative examination of swine sera for antibody to Aujeszky virus with the conventional and modified virus-serum neutralization test and a modified complement fixation test. Acta Veterinaria Scandinavia 17, 142-145.

MacFerran J. B. & Dow, C. (1975). Studies on immunisation of pigs with the Bartha strain of Aujeszkyts disease virus. Research in Veterinary Science 19, 17-22.

Kimman, T. G., Brouwers, R. A. M., Daus, F. J., Van Oirschot, J. T. & van Zaane, D. (1992). Measurement of isotype-specific antibody responses to Aujeszky's disease virus in sera and mucosal secretions of pigs. Veterinary Immunology & Immunopathology 31, 95-113.

Kimman, T. G., De Bruin, T. M. G., Voermans, J. J. M., Peeters, B. P. H. & Bianchi, A. T. J. (1995). Development and antigen specificity of the lymphoproliferation response to pseudorabies virus: dichotomy between secondary B- and T-cell responses. Immunology 86, 372-378.

Stellman, C., Vannier, P., Chappuis, G., Brun, A., Dauvergne, M., Fargeaud, D., Bugand, M. & Colson, X. (1989). The potency testing of pseudorabies vaccines in pigs. A proposal for a quantitative criterion and a minimum requirement. Journal of Biological Standardardization 17, 17-27.

Van Rooij, E. M. A., Haagmans, B. L., De Visser, Y. E., De Bruin, M. G. M., Boersma, W. & Bianchi, A. T. J. (1998). Effect of vaccination route and composition of DNA vaccine on the induction of protective immunity against pseudorabies infection in pigs. Veterinary Immunology & Immunopathology 66/2, 113-126.

Wardlaw, A. C. (1985). Practical Statistics for Experimental Piologists (19985). John Wiley & Sons. ISBN.0 471 90737 5.

Hilgers, L. A., Snippe, H. (1992). DDA as an immunological adjuvant. Research in Immunology 143(5), 494-503.

Gregoriadis, G., Saffie, R., De Souza, J. B. (1997). Liposome-mediated DNS vaccination. FEBS Letters 402, 107-110.

Ishii, N., Fukushima, J., Kaneko, T., Okada, E., Tani, K., Tanaka, S-I., Hamajima, K., Xin, K-Q., Kawamoto, S., Koff, W., Nishioka, K., Yasuda, T., Okuda, K. (1997). Cationic liposomes are strong adjuvants for a DNA vaccine of human immunodeficiency virus type 1. Aids Research and Human retroviruses 136, 1421-1428.

TABLE 1
Duration of virus excretion, fever and clinical signs
(mean No. days @ standard error of the mean) after
challenge infection with PRV strain NIA-3.

| Pig* | | Virus | Fever | Clinical | |
|---|---|---|---|---|---|
| groups | Mortality | excretion | (Tz40° C.) | signs | —G |
| gB/gD | 0/5 | 6.4² (0.3) | 2.4 (1.0) | 4.2² (0.2) | 1.25 |
| gB/gD + DDA | 0/5 | 2.8¹ (1.0) | 1.0³ (0.6) | 0.0¹ (0.0) | 2.01 |
| Control | 1/5 | 9.3 (0.3) | 5.0 (0.7) | 8.3 (1.3) | |
| plasmid | | | | | |

*Pigs that died from PRV challenge infection were excluded from the calculations.
¹Pigs vaccinated with gB/gD + DDA excreted virus for a significantly (P < 0.05) shorter period of time and suffered clinical signs for a significantly (P < 0.05) shorter period of time than pigs vaccinated with gB/gD or sham-vaccinated pigs.
²Pigs vaccinated with gB/gD excreted virus for a significantly (P < 0.05) shorter period of time and suffered clinical signs for a significantly (P < 0.05) shorter period of time than sham-vaccinated pigs.
³Pigs vaccinated with gB/gD + DDA had for a significantly (P < 0.05) shorter period of time over than shame-vaccinated pigs.

1. Use of a dimethyldialkylammonium salt as an adjuvant for a nucleic acid vaccine.

2. Use according to claim 1, wherein the dimethyldialkylammonium salt is a bromide, iodide or chloride salt.

3. Use according to claim 1 or 2, wherein the alkyl groups of the dimethyldialkylammonium salt are independently chosen from the group of unsaturated and saturated aliphatic alkyl chains having from 12 to 24, preferably 14 to 20 carbon atoms.

4. Nucleic acid vaccine comprising a nucleic acid and a dimethyldialkylammonium salt as adjuvant.

5. Vaccine according to claim 4, wherein the nucleic acid is a DNA, a cDNA, a positive or negative stranded RNA or mRNA molecule or a combination thereof.

6. Vaccine according to claim 4 or 5 in the form of a buffered salt solution.

7. Vaccine according to claim 6 wherein the dimethyldialkylammonium salt is present in an amount of between 0.5 and 32 mg, preferably between 1 and 16 mg per ml saline solution.

8. Vaccine according to any of the preceding claims, wherein the nucleic acid is present in a viral or bacterial delivery system.

9. Vaccine according to claim 8, wherein the nucleic acid is present in a Semliki or Sindbis virus delivery system.

10. Vaccine according to claim 8, wherein the nucleic acid is present in a Listeria monocytogenes bacterial delivery system.

11. Use of a dimethyldialkylammonium salt for enhancing the transfection and/or immune response of a nucleic acid vaccine.

12. Method of preparing a vaccine comprising formulating a composition of a dimethyldialkylammonium salt and a nucleic acid.

13. Method of invoking an immune response in a living organism by administration to said organism of a vaccine according to any of the claims 4-10.
